(19) United States
(12) Patent Application Publication
(10) Pub. No.: US 2016/0364822 A1
(43) Pub. Date: Dec. 15, 2016

(54) SYSTEM FOR ANALYZING DATA GATHERED FROM DEVICES AND OTHER SOURCES AND PROVIDING THRESHOLDS, ALERTS, AND/OR RECOMMENDATIONS TO GUIDE PREVENTION OF NEGATIVE WORKER OCCUPATIONAL HEALTH OUTCOMES

(71) Applicants: Monika Maya Wahi, Boston, MA (US); Katherine E. Arline, Cambridge, MA (US)

(72) Inventors: Monika Maya Wahi, Boston, MA (US); Katherine E. Arline, Cambridge, MA (US)

(21) Appl. No.: 15/137,587

(22) Filed: Apr. 25, 2016

Related U.S. Application Data
(60) Provisional application No. 62/175,570, filed on Jun. 15, 2015.

(51) Int. Cl. G06Q 50/26; A61B 5/00; G08B 6/00; A61B 5/16; G08B 5/00; G08B 3/00

(52) U.S. Cl. CPC G06Q 50/265; G08B 5/00; G08B 3/00; G08B 6/00; A61B 5/165; A61B 5/4519; A61B 2503/20

(57) ABSTRACT

This invention is a system to leverage worker data analytics to provide intelligence to decision-makers in the employment space to help optimally manage worker activities to prevent negative health-related outcomes associated with occupational conditions. This is done by obtaining data from a device worn on the worker, combining these data with other data, performing analytics, and providing intelligence to those in the employment space regarding whether worker has exceeded or is likely to exceed a threshold associated with negative occupational health-related outcomes. The system provides displays, alerts, and recommendations to those in the employment space to enable them to make informed decisions regarding protection of the worker's health.

### CROSS-REFERENCE TO RELATED APPLICATIONS

The present application claims the benefit of priority to U.S. Provisional Patent Application No. 62/175,570, filed Jun. 15, 2015, titled “A SYSTEM FOR ANALYZING DATA GATHERED FROM DEVICES AND OTHER SOURCES AND PROVIDING THRESHOLDS, ALERTS, AND/OR RECOMMENDATIONS TO GUIDE PREVENTION OF NEGATIVE WORKER OCCUPATIONAL HEALTH OUTCOMES”, which is hereby incorporated by reference in its entirety.

### FIELD OF THE INVENTION

According to the United States Bureau of Labor Statistics, there were 3,007,300 recorded cases of injuries, illnesses, and fatalities at work in 2013, resulting in 917,100 days away from work and 4,585 fatalities. Efforts to mitigate these outcomes are largely left up to employers and government agencies.

Data analytics can be leveraged using information gathered from workers to identify workers who are at a high likelihood for negative occupational health-related outcomes, and therefore can offer an opportunity for the employer or worker to prevent negative health-related outcomes associated with occupational conditions including but not limited to physical injury, burnout, muscle fatigue, overexertion, negative mental health outcomes such as anxiety and depression, and exhaustion.

### BACKGROUND OF THE INVENTION

This invention is a system to leverage data gathered from workers to provide intelligence to decision-makers in the employment space to help optimally manage worker activities to prevent negative health-related outcomes associated with occupational conditions. In this document, the word “worker” will be used to mean any paid or unpaid employee or volunteer of an organization following a defined role in the organization.

### BRIEF SUMMARY OF THE INVENTION

The purpose of the invention is to use data about a worker gathered from multiple sources including devices to determine if a worker has exceeded or is likely to exceed a biological, psychological, or other type of threshold associated with negative health-related outcomes associated with occupational conditions and provide this information to the worker and/or employer to enable them to make informed and optimal decisions regarding protection of the worker's health.

Data will be collected from a device or devices that are either attached to clothing worn by the worker, embedded in a piece of technology worn by the worker, embedded in clothing worn by the worker, or somehow otherwise attached to the worker, including through biological implantation. The device may include a sensor or multiple sensors. Data from this device or devices will be copied, transferred, or otherwise transmitted to a virtual or physical location or environment including but not limited to an application environment. This transmission may take place via bluetooth, WiFi, or any other mechanism.

The device data transmitted to the virtual or physical location or environment will be combined with data that relates to the worker that has been gathered from another source or other sources. These sources include but are not limited to other devices, other transmission locations on the same device, manual data entry, electronic data transfer, the employer, the worker, public sources, or any other source. The combined dataset will be processed through evidence-based algorithms to determine if a worker has exceeded or is likely to exceed a biological, psychological, or other type of threshold associated with negative occupational health-related outcomes including but not limited to physical injury, burnout, muscle fatigue, overexertion, negative mental health outcomes such as anxiety and depression, and exhaustion.

A software or some other type of interface will be provided to the worker so the worker can securely access displays of his/her own data from the device (and possibly other data from the combined dataset as well as data from other sources). Note that the worker interface may be standalone or combined with other interfaces. Similarly, a software or some other type of interface will be provided so the employer can securely access data from the employer's workers' devices (and possibly other data from the combined dataset as well as data from other sources). Note that the employer interface may be standalone or combined with other interfaces. Other software or other types of interfaces may be developed for other audiences, such as administration, and these could be combined with other interfaces. The worker and employer interface will provide displays of data as well as other displays, and may also provide other functions. Displays may include text, figures, images, or any other type of visuals, or other types of media including but not limited to audio and haptic, and may be made on any surface and come from any physical or virtual source. If other interfaces are developed, they may also provide displays as well as other functions.

Along with the displays of data in the worker and employer functions, displays will be made indicating the relative location of biological, psychological, or other type of threshold associated the particular data displayed based on prior and/or current analytics. In the worker and employer interfaces, a function will be available to provide an alert to the worker, the employer, or some other party or entities, or multiple parties or entities, when a threshold is approached or exceeded. The alert may be in the form of a particular type of data display (passive alert), a technological message transmission such as an e-mail (active alert), a telecommunications transmission such as a text message (active alert), or some other type of visual, audio, or other type of communication signaling, and can be active or passive.

Depending upon characteristics of the alert or the threshold that was approached or exceeded, the software make or may not also provide a recommendation linked to an active alert during transmission, a passive alert provided in a display, any other alert, or to the threshold that was approached or exceeded. The recommendation may be communicated as part of the active alert transmission, the passive alert display, or may be communicated some other way to any entity or party designated by the employer. A recommendation is a suggested course of action associated with one or more thresholds that triggered the alert or that was approached or exceeded in the worker or workers that is communicated to the employer and/or employee and/or some other party or multiple parties. The course of action is suggested upon contingency of the approaching or exceeding of a particular threshold or the triggering of an alert linked to a particular threshold.

The contents of this summary section are provided only as a simplified introduction to the invention, and are not intended to be used to limit the scope of the appended claims. The present disclosure has been described above in terms of presently preferred embodiments so that an understanding of the present disclosure can be conveyed. However, there are other embodiments not specifically described herein for which the present disclosure is applicable. Therefore, the present disclosure should not be seen as limited to the forms shown, which should be considered illustrative rather than restrictive.

### BRIEF DESCRIPTION OF THE DRAWINGS

Other systems, methods, features and advantages of the present invention will be or will become apparent to one of ordinary skill in the art upon examination of the following figures and detailed descriptions. It is intended that all such additional apparatuses, systems, methods, features and advantages be included within this description, be within the scope of the present invention, and be protected by the appended claims. Component parts shown in the drawings are not necessarily to scale, and may be exaggerated to better illustrate the important features of the present invention. In the drawings, like reference numerals designate like parts throughout the different views.

FIG. 1. Worker shift data.

FIG. 2. Combined dataset development and active alerts.

FIG. 3. Worker interface.

FIG. 4. Employer interface.

### DETAILED DESCRIPTION OF THE INVENTION

FIG. 1 shows a flow of data collected specifically about the worker's shift. The worker wears the device to collect the data during his/her shift. The device could measure anything, but examples include the following: Fitbit®, Misfit®, Garmin®, Armour Band®, Microsoft Band®, Xiaomi® steps tracking devices (which may also track heartbeat, sleep, and swimming, depending upon the model), fitness tracking systems such as Jawbone® which allow user input of data such as meals and mood, a smartphone (such as iPhone and Android) used as a tracking device, and smart watches that track activity such as Apple Watch®, Mondaine®, and Withings®. At 101, the beginning of the shift, the device is deployed to collect data, and at 103, the end of the shift, the device is undeployed. Also, note is made about the shift start [102] and stop times [104]. This notation may be made manually or automatically, but must become electronic data before it can be used in the system.

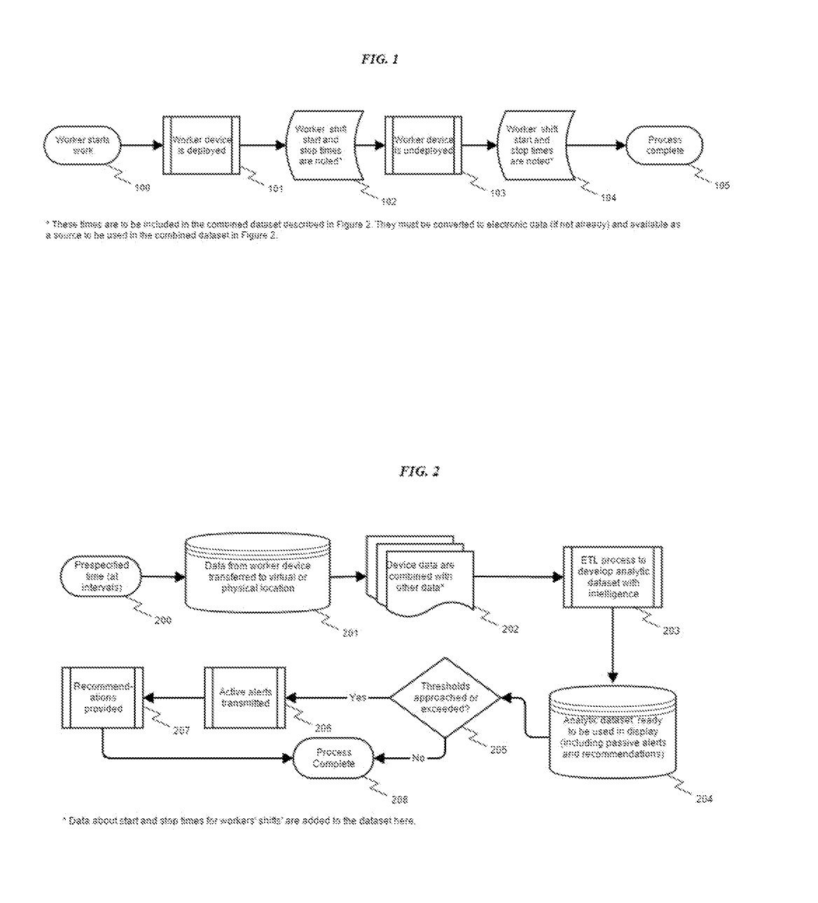

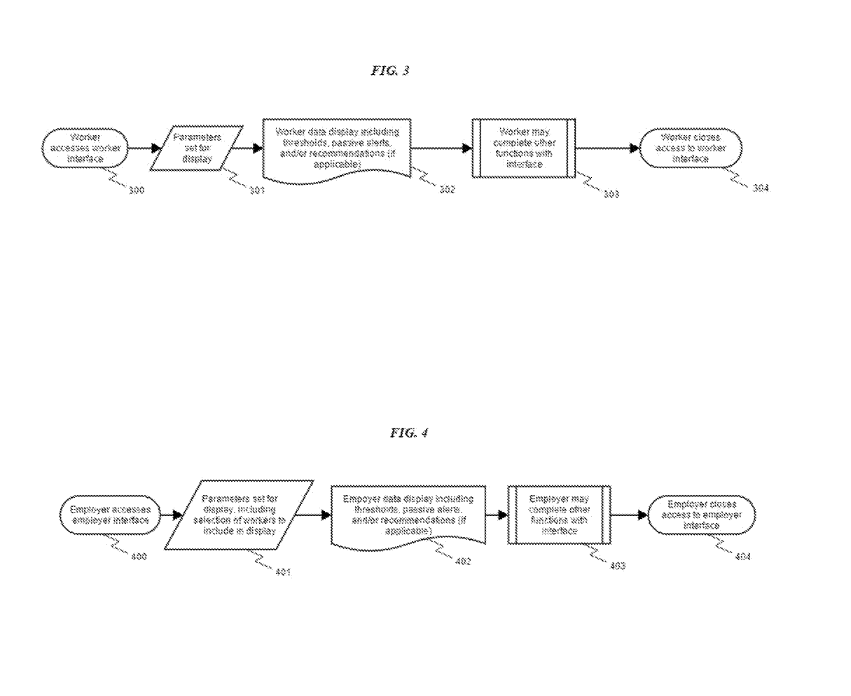

FIG. 2 shows how both the device data and the shift data are entered into the system and are combined with other data. First, at prespecified time intervals, device data are transferred from the device to the virtual or physical location [201]. There, device data are combined with other data, including the shift data [202]. The other data will include both static and time-dependent predictors of negative occupational health-related outcomes, which are hypothesized to include the following: stressful work environment, lack of worker experience in the occupation, worker being in a state of burnout, worker being physically ill, worker being mentally ill, worker lacking adequate functional information and training, worker participation in certain high-risk occupations, worker demographics, worker locus of control level role, underlying worker personality components, underlying worker physical and mental co-morbidities, and quality and nature of management in the worker's organization. The combined dataset undergoes extract, transform, and load (ETL) processing [C] to add intelligence (including but not limited to threshold information, alert information, recommendation information, and other calculations and algorithms). This processed dataset is called analytic dataset, and after ETL processing, it is ready to display thresholds, passive alerts, and recommendations when a worker, employer, or anyone else accesses an interface and chooses a display.

After the analytic dataset is prepared, logical operations [205] will be run on it to determine if predefined evidence-based thresholds associated with negative health-related outcomes associated with occupational conditions are approached or exceeded. If no thresholds are being approached or exceeded according to the logical operations, the process is complete [208]. On the other hand, if pre-defined active alerts have been programmed, they will be triggered [206], and if recommendations are available and are desired, they will be provided [207] before the process is completed [208].

FIG. 3 shows the worker interface. The worker accesses the secure interface and then sets parameters for display, including but not limited to time frame that will be the subject of the display [301]. Next, worker accesses data display, which can include thresholds, passive alerts, and/or recommendations (if applicable) [302]. The interface will allow the worker to complete other functions before the worker closes access to the interface.

FIG. 4 shows the employer interface. As with the worker interface, the employer will access the secure interface and set parameters for display [401], then access the display [402]. Two main differences between the employer and worker interface include 1) the employer will be asked to set the additional parameter as to which employees to display (and may be asked for other parameters), and 2) the employer display will include thresholds, passive alerts, and recommendations for all the employees in the parameter selected. Recommendations provided in the employer interface may also be different than ones displayed in the worker interface. As with the worker interface, the employer may also complete other functions in the interface [403] before closing access to the interface.

### EXEMPLARY VERSION OF THE INVENTION

In an exemplary version of the invention, the employer would provide or be provided worker devices as part of this system, and assign the various devices to workers (such as nurses). A typical device that could be used currently would be one embedded in a wearable health tracking device that tracks biological metrics that is already available on the market (such as a pedometer). The employer would track device assignment in the employer interface. This employer interface would be provided through secure online software on the internet that could be accessed through a computer or portable electronic device such as smartphone or tablet. In the employer interface, the employer would log in and do data entry to record which employee had which device. The employer would also develop administrative policies regarding use of the system in the employment space and communicate this to workers when the device is assigned to them.

The devices would be easily attachable to the clothing or body of the worker, and would be put on and removed at the beginning and end of shifts by the worker. The worker interface would also be secure, accessed as software through the internet by the worker, and would work on a computer or portable electronic device such as smartphone or tablet. As part of administrative policies, the worker would be expected to wear the device while working, and access the worker interface to perform functions.

The data from the device would be transmitted securely through the internet to secure storage that permits access by both customers and developers. Also in this storage environment would be applications to allow analytics where the data would be combined with other data and the analytic dataset would be developed. The data storage environment would support the other functions of the system, such as data displays, data entry, and other processes.

Providing data to the system about actual shift times would be best done through manual data entry either by the employer or worker through their respective interfaces. In the case where last-minute changes in shift times are common, it may be better to have the worker enter these data because they will be more accurate. The data could be collected in the storage environment to be combined with other data.

When workers access their secure interface, the displays they are provided will be shown as online reports of their data. They would be provided information about their data combined with other data about their shifts (such as mean steps taken per shift). The data would be reported as clearly labeled numbers, but also provided in a graphical display, such as line or bar charts. The interface would allow the worker to set parameters around the display, such as selecting which shift's data to display, or what type of graphical display is desired.

When employers access their secure interface, they will have the same functionality as the worker with respect to displays, except they will have the additional functionality associated with being able to view the data from multiple workers (such as a nursing team). Data displays will be similar to the ones in the worker interface, but will allow for the inclusion of multiple workers. Like the worker interface, the employer interface would allow the employer to set parameters around the around the display, such as selecting which workers' data to display, which shift's data to display, or what type of graphical display is desired.

If a threshold has been determined through research and development on the data from the device and the occupation of the worker that has been shown through scientific evidence to be increase the likelihood of negative health-related outcomes associated with occupational conditions, this threshold will be included in data displays to the worker and the employer in their interfaces (such as a line across a bar graph showing average number of steps per shift indicating the average steps for shift threshold found to be associated with negative health-related outcomes associated with occupational conditions).

Accompanying the display of thresholds will be passive alerts. Through administrative policies, under what conditions passive alerts are displayed on the interface (and to whom) will be set, but best practices would suggest they be set to be displayed to both worker and employer 1) when the worker is approaching the threshold, and 2) when the worker exceeds the threshold. A passive alert will provide an explanation as to why the alert was triggered relating it to the threshold, such as a text box on the display explaining that a worker is approaching or exceeding a threshold (e.g., “Healthy level of steps for shift exceeded”).

In addition, active alerts can be used in this system. These would be best implemented by having the employer use the employer interface to designate under what conditions to be sent active alerts. Active alerts would contain the same information as passive alerts, but would be transmitted in real-time as soon as the alert is created. The alert will be created when the data that is transferred into the storage environment is analyzed, and thus the alert is created. As a passive alert, it will not be seen until the worker or employer uses their respective displays, but as an active alert, a message could be actively transmitted at this time (such as the employer receiving the alert information in his/her e-mail).

In addition, recommendations can be provided by the system to the employer or worker. Recommendations can accompany passive or active alerts. For example, when an employer receives an alert that a worker is approaching a threshold, the employer may also receive the recommendation not to schedule that worker again for a particular time period to prevent endangering the worker's health.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted.

Having described our invention, we claim:

1. A system for analyzing data gathered from devices and other sources and providing thresholds, alerts, and/or recommendations to guide prevention of negative worker occupational health outcomes comprising:
worker devices attached in some manner to a worker for obtaining data;
a processor for combining the data with other data; and
interfaces including displays and transmission of active alerts for providing evidence-based thresholds, alerts, or recommendations to guide prevention of negative worker occupational health outcomes.

2. The system as recited in claim 1, wherein the data from at least one worker device is combined with the other data.

3. The system as recited in claim 1, wherein the interfaces comprise at least one secure worker interface to allow the worker to access worker data.

4. The system as recited in claim 1, wherein the interfaces comprise at least one secure employer interface that allows an employer to access worker data.

5. The system as recited in claim 1, wherein analytics take place between combining the data and providing interface displays for providing the evidence-based thresholds, alerts, or recommendations.

6. The system as recited in claim 1, wherein the thresholds provided to the system are produced through research and development using the worker device that is being used in the system, with the research and development based on a same occupational group that is being served by the system.

7. The system as recited in claim 1, wherein the purpose of the system is to provide guidance and intelligence to provide information to those in an employment space including but not limited to the worker and the employer so the information can be used for protecting the worker's health.

8. The system as recited in claim 1, wherein the worker comprises any paid or unpaid employee or volunteer of an organization following a defined role in the organization.

9. The system as recited in claim 1, wherein the negative worker occupational health outcomes includes but is not limited to physical injury, burnout, muscle fatigue, overexertion, negative mental health outcomes such as anxiety and depression, and exhaustion.

10. The system as recited in claim 1, wherein the displays comprise text, figures, images, or any other type of visuals, or other types of media including but not limited to audio and haptic, and may be made on any surface and come from any physical or virtual source.

11. The system as recited in claim 1, wherein the interface comprises a software interface or some other type of interface.

12. A method for analyzing data gathered from devices and other sources and providing thresholds, alerts, and/or recommendations to guide prevention of negative worker occupational health outcomes comprising:
obtaining data from devices attached in some manner to a worker;
combining the data with other data; and
providing evidence-based thresholds, alerts, or recommendations to guide prevention of negative worker occupational health outcomes using interfaces including displays and transmission of active alerts.
